SEQUENCE LISTING (7 sequences)

SEQ ID NO: 1 - DNA, 30 bp, Artificial
  PCR primer
ataagatctggatccatgcacaccatgtcc

SEQ ID NO: 2 - DNA, 37 bp, Artificial
  PCR primer
tctagagaattcctacatcctgtagttcttgtttcct

SEQ ID NO: 3 - DNA, 17 bp, Homo sapiens
atgcacaccatgtcctc

SEQ ID NO: 4 - DNA, 20 bp, Homo sapiens
catcctgtagttcttgtttc

SEQ ID NO: 5 - DNA, 18 bp, Artificial
  PCR primer
atggggggttctcatcat

SEQ ID NO: 6 - DNA, 20 bp, Homo sapiens
tttcaacaagcccacagggt

SEQ ID NO: 7 - DNA, 19 bp, Homo sapiens
ccacgatgcctgtctgagg

(19) United States
(12) Patent Application Publication
(10) Pub. No.: US 2003/0186918 A1
(43) Pub. Date: Oct. 2, 2003

(54) Increasing growth factor production by cells

(75) Inventor: Edward Chaum, Germantown, TN (US)

Correspondence Address: Margaret J. McLaren, Ph.D., Esq., Akerman Senterfitt, Suite 400, 222 Lakeview Avenue, West Palm Beach, FL, 33402-3188, US

(73) Assignee: The University of Tennessee Research Corporation, Memphis, TN

(21) Appl. No.: 10/397,933

(22) Filed: Mar. 26, 2003

Related U.S. Application Data
(60) Provisional application No. 60/367,873, filed on Mar. 27, 2002.

(51) Int. Cl. A61K 48/00

(52) U.S. Cl. 514/44; 424/93.21

(57) ABSTRACT

Disclosed are methods and compositions for growth factor gene therapy for conditions involving degeneration or injury of cells of the retina, including age-related macular degeneration. Included in the invention are non-viral vectors for delivery of growth factor fusion proteins, cells transduced with such vectors, and methods of treatment using these vectors.

### CROSS REFERENCE TO RELATED APPLICATIONS

This application claims priority from U.S. Provisional Application Serial No. 60/367,873 filed Mar. 27, 2002. The foregoing is incorporated herein by reference.

### STATEMENT REGARDING FEDERALLY SPONSORED RESEARCH

This invention was made with U.S. government support under grant number K08 EY00381 awarded by the National Institutes of Health. The U.S. government may have certain rights in the invention.

### FIELD OF THE INVENTION

This invention relates generally to the fields of molecular biology and medicine. More particularly, the invention relates to methods of gene therapy for eye diseases using growth factors.

### BACKGROUND

The retinal pigment epithelium (RPE) serves many critical functions in maintaining the health of the neurosensory retina. One of these functions is the production of growth factors (also known as cytokines, neurotrophic factors, and trophic hormones), that have both paracrine and autocrine activity in the RPE (Waldbillig R J, et al. J Neurochem 1991, 57:1522-1533; Takagi H, et al., Invest Ophthalmol Vis Sci 1994, 35:916-923; Schweigerer I, et al., Biochem Biophys Res Commun 1987, 143:934-940; Martin D M et al., Brain Res Mol Brain Res 1992, 12:181-186).

Insulin-like growth factor-1 (IGF-1), basic fibroblast growth factor (bFGF) and other growth factors have been shown to stimulate DNA synthesis and RPE cell proliferation in vitro (Martin D M et al., Brain Res Mol Brain Res 1992, 12:181-186; Campochiaro P A, et al., J Cell Sci 1994,107:2459-2469; Gupta S K et al., Biochem Cell Biol 1997, 75:119-125), and to participate in the responses to injury of RPE cells and retinal neurons (Cao W, et al., Exp Eye Res 2001, 72:591-604).

Growth factors (Faktorovitch E G, et al., Nature 1990, 347:83-86; Uteza Y, et al., Proc Natl Acad Sci USA 1999, 96:3126-3131; Unoki K et al., Invest Ophthalmol Vis Sci 1994, 35:907-915; Cayouette M, and Gravel C, Hum Gene Ther 1997, 8:423-430; Frasson M et al., Invest Ophthalmol Vis Sci 1999, 40:2724-2734; Lau D, et al., Invest Ophthalmol Vis Sci 2000, 41:3622-3633) and secondary messengers of the intracellular signaling pathways stimulated by these factors (Allsopp T E et al., Cell 1993, 73:295-307; Chen J et al., Proc Natl Acad Sci USA 1996, 93:7042-7047; Joseph R M, and Li T, Invest Ophthalmol Vis Sci 1996, 37:2434-2446; Kermer P et al., J Neurosci 2000, 20:722-728) have been shown to have neuroprotective effects on retinal neurons in animal models of induced and genetic retinal degeneration, and similarly may play a protective role in human retinal degenerative diseases.

There is a growing body of evidence that signaling cascades resulting from stimulation of growth factors may result in anti-apoptotic activity in the neurons of the CNS, and may play a comparable neuroprotective role in the retina (Allsopp T E et al., Cell 1993, 73:295-307; Chen J et al., Proc Natl Acad Sci USA 1996, 93:7042-7047; Joseph R M, and Li T, Invest Ophthalmol Vis Sci 1996, 37:2434-2446; Kermer P et al., J Neurosci 2000, 20:722-728; Párrizas M, et al., J Biol Cheml997, 272:154-161; Peruzzi F et al., Mol Cell Biol 1999, 19:7203-7215; Ye P, et al., Endocrinology 1999, 140:3063-3072; Seigel GM et al., Mol Vis. 2000, 6:157-163).

Age-related macular degeneration (AMD) is the leading cause of blindness among the elderly population. The cause of the condition is presently unknown, but it is believed to involve a large number of genes, making it unsuitable for therapies designed to target a unique pathogenic entity, such as single defective gene product. Presently, there is no effective therapy to prevent or slow the progression of this disease. Currently, clinical management of this disease is primarily limited to attempts, such as laser photocoagulation, to control devastating growth of new blood vessels into the retina that occurs in the so-called “wet” form of AMD.

For reasons not yet understood, the cell type primarily affected in AMD is the RPE. Among their various functions, RPE cells are highly active phagocytes. On a daily basis, they are required to engulf and digest thousands of lipid-rich disc membranes shed from the outer segments of the photoreceptors. Not unexpectedly, with advancing age, breakdown products of cellular metabolism begin to accumulate within and beneath the RPE cells, impairing their function. These deposits include the aging pigment lipofuscin, as well as nodular deposits known as drusen. The latter deposits are notably abundant in patients with both wet and dry forms of AMD, and are considered the hallmark of the disease.

As the aged increasingly dominate the world population, there exists a strong need for improved methods for prevention and treatment of AMD. In particular, new strategies are needed to improve the capacity of aging RPE cells to protect themselves, and the neighboring retinal and choroidal cells they support.

### SUMMARY

The invention relates to methods and compositions for enhancing the survival, proliferation and growth factor synthetic capability of RPE cells, by stimulating growth factor expression in these cells. In particular, it relates to targeted gene therapy using growth factor transgenes, to enhance the expression by the RPE of factors having trophic effects on the RPE, retina and choroid.

Transduced human RPE cells expressing a human growth factor (i.e., IGF-1) transgene were produced and shown to express high levels of IGF-1 MRNA, and to synthesize and secrete high levels of an IGF-1 fusion protein into the tissue culture media. Compared to controls, transduced clones demonstrated a dose-dependent, enhanced ability to proliferate under conditions of stress (i.e., under low serum conditions). Clones that expressed moderate and high levels of the IGF-1 fusion protein grew at a significantly faster rate than controls. The biological effect of the fusion protein was to increase recruitment of G₀-G₁ phase cells into the proliferative phase of the cell cycle. Expression and secretion of the IGF-1 transgene enhanced growth characteristics in a dose-dependent manner, and modulated the proliferative potential of the RPE cells. Collectively, these findings demonstrate the biologic activity of a growth factor fusion protein engineered to be synthesized and secreted by human RPE cells, and show that increasing expression of a growth factor using gene transfer can effect changes in the growth rate and cell cycle kinetics of human RPE cells.

Production of such factors could enhance the survival and function of the RPE by autocrine mechanisms. Furthermore, secretion of these factors by the RPE would provide enhanced levels of trophic factors to the neighboring tissues adjacent to the RPE, i.e., the cells of the neurosensory retina, (particularly the photoreceptors), and of the choroid, the source of the sight-destroying abnormal new blood vessels that grow through the RPE to enter the retina in the wet form of AMD.

Accordingly, in one aspect the invention features a method of increasing synthesis or secretion of a growth factor protein by a RPE cell. The method includes the steps of: (a) providing a RPE cell; and (b) transducing the cell with an expression vector containing a growth factor-encoding nucleic acid, in an amount effective to increase synthesis or secretion of the growth factor protein. The growth factor protein can include at least one of IGF- 1, bFGF, aFGF, CNTF, PDGF, and BDNF. In a preferred embodiment of the method, the growth factor protein is human IGF-1.

Some embodiments of the method include use of expression vectors containing nucleic acids that express growth factor proteins in the form of fusion proteins. Preferred embodiments of growth factor fusion proteins can include an epitope tag, for example, a His₆ tag, that facilitates the isolation and identification of the expressed protein. A particularly preferred embodiment of an epitope-tagged growth factor fusion protein is His₆-tagged human IGF-1.

In yet another embodiment of this method, the expressed growth factor fusion protein can include a reporter protein. The reporter protein can be, for example, green fluorescent protein (GFP), luciferase, or β-galactosidase. A preferred embodiment of a growth factor fusion protein fused with a reporter protein useful in the invention is human IGF-1 tagged with GFP.

In some embodiments of the method, the expression vector can include an inducible promoter.

In another aspect, the invention provides an expression vector including a growth factor-encoding nucleic acid, wherein the vector directs the production of an expressed growth factor fusion protein. In particular embodiments of the vectors, the fusion protein can be human IGF-1. Preferred embodiments of the vector can be non-viral vectors, for example, plasmid vectors.

The invention further provides a kit for gene transduction of a cell. The kit can include at least one expression vector containing a nucleic acid encoding a human growth factor protein fused with a reporter protein, or a nucleic acid encoding a human growth factor protein fused with an epitope tag, and instructions for use. In preferred embodiments of the kit, at least one expression vector directs expression of a human IGF-1 protein linked to GFP as the reporter. In other preferred embodiments, at least one expression vector directs expression of a human IGF-1 fusion protein linked to a His₆ tag.

Also encompassed by the invention are retinal cells transduced with the vectors of the invention. Preferred cells used for transduction by the vectors are RPE cells, particularly human RPE cells.

The invention further provides a method of treating dysfunction or injury of a RPE cell. The method includes transducing the RPE cell with an expression vector that expresses a growth factor protein in an amount sufficient to improve or cure the dysfunction or injury. The method can be used to treat dysfunction affecting the RPE cell that is caused by conditions such as cancer, viral infection, diabetes, hereditary RPE dystrophy, AMD, retinitis pigmentosa or drug-induced toxicity.

The method can also be used to treat dysfunction affecting the RPE that involves inability to divide, or commitment to programmed cell death.

Injuries of the RPE cells amenable to treatment by the method can include, for example, those caused by abrasions, ocular trauma, surgical and laser procedures, or retinal detachment.

The invention further includes a method of treating a retinal disease or condition. The method includes the steps of: (a) providing a subject having or at risk for developing a retinal disease or condition; and (b) providing an expression vector including a growth factor-encoding nucleic acid; and (c) administering to the subject an amount of the vector sufficient to ameliorate or cure the disease or condition. Retinal diseases and conditions that can be treated by the method can include cancer, viral infections, diabetic retinopathy, hereditary RPE dystrophies, AMD, retinitis pigmentosa, drug-induced toxicity, macular commotio, and surgical- and laser-induced injuries. In preferred embodiments of the method, the growth factor is human IGF-1.

Unless otherwise defined, all technical terms used herein have the same meaning as commonly understood by one of ordinary skill in the art to which this invention belongs.

As used herein, the term “growth factor” refers to a protein hormone that has numerous cellular functions. These functions can include, but are not limited to: 1) stimulating or promoting cell division; 2) up- or down-regulating expression of other genes; inhibiting programmed cell death (apoptosis); 4) stimulating or regulating cell migration and motility; 5) participating in cell differentiation, that is, development into progressively more specialized cell types; and f) activating intracellular signaling pathways. Typical growth factors, known to effect one or more of these functions in cells of the retina, including RPE cells and photoreceptors, include such factors as IGF-1, bFGF, acidic fibroblast growth factor (aFGF), ciliary neurotrophic factor (CNTF), platelet derived growth factor (PDGF), and brain-derived growth factor (BDNF).

The term “epitope tag,” as used herein, refers to a short sequence of amino acids added to a protein, for example a transgenic protein, that permits the protein to be identified using a method that specifically binds to the tag. Identification methods can include, for example, use of epitope-specific antibodies or column chromatography. An epitope tag on a transgenic protein allows the transgenic protein to be distinguished from the corresponding naturally produced protein.

The term “subject,” as used herein, means a human or non-human animal, including but not limited to a mammal such as a dog, cat, horse, cow, pig, sheep, goat, chicken, primate, rat, or mouse.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions, will control. In addition, the particular embodiments discussed below are illustrative only and not intended to be limiting.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic diagram showing a pcDNA:IGF-1 fusion gene vector. A human IGF-1 coding sequence is inserted into the multiple cloning site (MCS) fused with a short upstream His₆Xpress-epitope sequence, under the control of a cytomegalovirus (CMV) promoter. The selectable neomycin-resistance gene (neo^{r}) is under the control of a simian virus (SV40) promoter. Arrows indicate the location of RT-PCR primers used for differential amplification of both intrinsic and transgenic IGF-1 message (primer pair P1, P2), or transgenic IGF-1 message only (primer pair P2, P3).

FIG. 2 illustrates semi-quantitative RT-PCR showing expression of the IGF-1 transgene. (A) Band shows amplification of intrinsic and transgenic IGF- 1 transcripts in transduced clones c49, c14 and c62. Both transcripts are detected using primers 1 and 2 (see FIG. 1). IGF-1 is barely detectable in untransduced controls (RPE). (B) High levels of transgenic mRNA are detected in the transduced clones using transgene-specific primers 2 and 3. These primers amplified no transgenic message in untransfected control cells (RPE). (C) Amplified β-actin message from the same RNA samples.

FIG. 3 is a Western blot showing IGF-1 synthesis in untransfected RPE cells and transduced clones. The transduced clones demonstrate moderate (c14) and high (c62) levels of IGF-1 fusion protein synthesis, relative to naive control cells (RPE). The first lane was loaded with recombinant human IGF-1 protein. The Western blot on the right demonstrates the higher molecular weight of the IGF-1 fusion protein present in clone 62, compared with recombinant human IGF-1.

FIG. 4 is a Western blot showing transgenic IGF-1 fusion protein secretion. Lane 1 (IGF-1): recombinant human IGF-1 protein; lane 2 (c62): secreted IGF-1 protein in fourfold concentrated medium conditioned by clone c62; lane 3 (RPE): fourfold concentrated medium conditioned by naive RPE cells; lane 4(c62, Ni-TED): protein eluted from a nickel column loaded with medium conditioned by clone c62. Result in lane 4 confirms the presence of a His₆-tag on the IGF-1 fusion protein secreted into the medium.

FIG. 5 is a graph showing cloned RPE cell proliferation in low serum. Over 6 days, IGF-1 transduced clones, and IGF-1 -treated RPE cells showed an enhanced growth rate compared with control cells. The increase in growth in the clones was IGF-1 dose dependent. There was no difference in the growth, compared with naive RPE control, of a G418-resistant clone that did not express a GFP:IGF-1 fusion protein. *Statistically significant: c62, P<0.002; c14, P<0.002; IGF-1-treated, P<0.035; paired samples, t-test.

FIG. 6 is three graphs showing flow cytometric analyses of RPE cells 27 hours after release from serum starvation. The number of cells that progressed from G₀-G₁ into S-phase and G₂—mitosis was significantly higher in IGF-₁—transduced clones c14 (P<0.045, paired samples t-test) and c62 (P<0.025) than in the control RPE cells. (Compare A, B and C.) The increase in cell cycle kinetics in the transduced clones was IGF-1 dose dependent.

### DETAILED DESCRIPTION

The invention encompasses compositions and methods relating to increasing expression of growth factors in cells of the retina, such as the RPE, using gene transfer techniques. The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, Ausubel et al. eds., Greene Publishing and Wiley-Interscience, New York, 1992 (with periodic updates). Various techniques using polymerase chain reaction (PCR) are described, for example, in Innis et al., PCR Protocols: A Guide to Methods and Applications, Academic Press: San Diego, 1990. PCR primer pairs can be derived from known sequences by known techniques, such as use of computer programs intended for that purpose (for example, Primer, Version 0.5, 1991, Whitehead Institute for Biomedical Research, Cambridge, Mass.). Methods for chemical synthesis of nucleic acids are discussed, for example, in Beaucage and Carruthers, Tetra Letts 22:1859-1862, 1981, and Matteucci et al., J Am Chem Soc 103:3185, 1981. Chemical synthesis of nucleic acids can be performed, for example, on commercial automated oligonucleotide synthesizers.

Immunological methods (for example, preparation of antigen-specific antibodies, immunoprecipitation, and immunoblotting) are described, for example, in Current Protocols in Immunology, Coligan et al., eds., John Wiley & Sons, New York, 1991; and Methods of Immunological Analysis, Masseyeff et al., eds., John Wiley & Sons, New York, 1992.

Conventional methods of gene transfer and gene therapy can also be adapted for use in the present invention. See, for example, Gene Therapy: Principles and Applications, T Blackenstein, ed., Springer Verlag, 1999; Gene Therapy Protocols (Methods in Molecular Medicine), P D Robbins ed., Humana Press, 1997; and Retro-vectors for Human Gene Therapy, C P Hodgson, ed., Springer Verlag, 1996.

Increasing Growth Factor Synthesis or Secretion by a RPE Cell

Prominent among the many functions of the RPE critical to maintaining the health of the neurosensory retina is the production of growth factors (trophic hormones). The invention provides a method of increasing growth factor synthesis or secretion by a RPE cell that includes transducing a RPE cell with an expression vector containing a nucleic acid that encodes a growth factor protein.

The method encompasses increasing synthesis or secretion of any growth factor known to have a trophic or neuroprotective effect on the retina. Suitable growth factors can include but are not limited to IGF-1, bFGF, aFGF, CNTF, PDGF and BDNF. Such factors are known to stimulate DNA synthesis and RPE cell proliferation in vitro. Growth factors are also involved in RPE and neuronal response to injury, having neuroprotective effects in animal models of neuronal degeneration.

Among the foregoing factors, IGF-1 may be particularly suitable for conditions, such as RPE dystrophies or injuries, in which proliferation of the RPE is desirable. IGF-1 is a potent stimulator of DNA synthesis in many cell types. A dose-dependent correlation between IGF-1 expression and cell proliferation rates has been shown in the central nervous system (Khandwala H M, et al., Endocr Rev 2000;21 :215-244), and is demonstrated in human RPE cells in examples described herein. IGF-1 is thought to exert its mitogenic effect via the mitogen-activated protein kinase (MEK/ERK) cascade. Activation of the MEK/ERK pathway by the binding of IGF-1 to its membrane-bound receptor kinase results in up-regulation of cyclin D1 expression, a factor necessary to traverse G₁ restriction, accounting in part for its role as a cell cycle progression factor (Rubin R, and Baserga R, Lab Invest. 1995, 73:311-331). As described herein, enhancement of entry into the cell cycle can be achieved by transduction of adult human RPE cells with a vector expressing a human IGF-1 fusion protein.

A further advantage of IGF-1 for growth factor gene therapy is its multipotency. IGF-1 receptor activation is known to result in specific phosphorylation of substrate proteins that participate in several molecular pathways that regulate diverse cellular processes. Among these processes is inhibition of apoptosis (Sasaoka T, et al., J Biol Chem 1994, 269:13689-13694; Blakesley V A et al., Contemporary Endocrinology: The IGF-1 System. Totowa, N.J., ed., Humana Press; 1999:143-163). IGF-1 acts as a potent survival factor for neuronal and glial cells by inhibiting apoptosis via the PI3-K signaling cascade (Ye P, et al., Endocrinology 1999, 140:3063-3072; Barres B A et al., Development 1993, 118:283-295; Yao D L et al., J Neurosci Res 1995, 40:647-659; Cardone M H et al., Science 1998, 282:1318-1321; Datta S R et al., Cell 1997, 91:231-241; Pugazhenthi S et al., J Biol Chem 2000, 275:10761-10766; KermerP et al., J Neurosci 2000, 20:2-8.) Increased expression of IGF-1 in RPE cells transduced with an IGF-1 expression vector may similarly effect such benefits as resistance to apoptosis. Additionally, as in other cell types (Heck S et al., J Biol Chem 1999, 274:9828-9835), IGF-1 may further provide protection of the RPE and other retinal cells against oxidative stress through PI3-K activation of nuclear factor-κB.

### Vectors for Expressing Growth Factors

Within the invention is a method of increasing synthesis or secretion or a growth factor that includes: (a) providing a RPE cell; (b) transducing the RPE cell with an expression vector that includes a growth factor-encoding nucleic acid.

Natural or synthetic nucleic acids encoding growth factors can be incorporated into recombinant nucleic acid constructs, typically DNA constructs, capable of introduction into and replication in a host cell. Such a construct preferably is a vector that includes a replication system and sequences that are capable of transcription and translation of a polypeptide-encoding sequence in a given host cell. For the present invention, conventional compositions and methods for preparing and using vectors and host cells can be employed, as described, for example, in Sambrook et al., supra, or Ausubel et al., supra.

Expression of a growth factor gene in a host cell (for example, one in an animal) is achieved by introducing into the host cell a nucleic acid sequence containing a growth factor gene encoding a growth factor polypeptide.

An “expression vector” is a vector capable of expressing a DNA (or cDNA) molecule cloned into the vector and, in certain cases, producing a polypeptide or protein. Appropriate transcriptional and/or translational control sequences are included in the vector to allow it to be expressed in a cell. Expression of the cloned sequences occurs when the expression vector is introduced into an appropriate host cell. If a eukaryotic expression vector is employed, then the appropriate host cell would be any eukaryotic cell capable of expressing the cloned sequences. As described below, in preferred embodiments of the invention, human retinal cells, and in particular human RPE cells, are appropriate host cells. Similarly, if a prokaryotic expression vector is employed, then the appropriate host cell would be any prokaryotic cell capable of expressing the cloned sequences. A number of vectors suitable for stable transformation of animal cells or for the establishment of transgenic animals are known. See, for example, Pouwels et al., Cloning Vectors: A Laboratory Manual, 1985, Supp. 1987. Vectors shown to be useful in the successful transduction of human RPE cells, leading to synthesis and secretion of growth factors by the transduced cells, are described in the examples below.

The precise nature of regulatory regions needed for gene expression may vary from organism to organism, but in general include a promoter which directs the initiation of RNA transcription. Such regions may include those 5′-non-coding sequences involved with initiation of transcription such as the TATA box. The promoter may be constitutive or regulatable. Constitutive promoters are those which cause an operably linked gene to be expressed essentially at all times. Regulatable promoters, by contrast, are those which can be activated or deactivated. Regulatable promoters include “inducible” promoters, which are usually “off” but which may be induced to turn “on,” and “repressible” promoters, which are usually “on,” but may be turned “off.” Many different regulatable promoters are known, including those regulated by temperature, hormones, heavy metals, the product of the gene, regulatory proteins, and antibiotics such as doxycycline. These distinctions are not absolute; a constitutive promoter may be regulatable to some degree. The regulatability of a promoter may be associated with a particular genetic element, such as an “operator,” to which an inducer or repressor binds. The operator may be modified to alter its regulation. Hybrid promoters may be constructed in which the operator of one promoter is transferred into another. Use of an inducible promoter could be beneficial, for instance for controlled expression of a growth factor, for example to permit expression of the factor by a RPE cell for a limited period of time.

The promoter may be a “ubiquitous” promoter active in essentially all cells of the host organism, for example, the β-actin promoter, or it may be a promoter whose expression is more or less specific to the target cells, for instance specific retinal cells such as RPE cells or photoreceptors. Preferably, the cell-specific promoters are essentially not active outside a given tissue type, such as the retina.

Typically, animal expression vectors include (1) one or more cloned animal genes under the transcriptional control of 5′ and 3′ regulatory sequences and (2) a dominant selectable marker. Such animal expression vectors may also contain, if desired, a promoter regulatory region (for example, a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

An example of a useful promoter which could be used to express a gene according to the invention is a cytomegalovirus (CMV) immediate early promoter (CMV IE) (Xu et al., Gene 272: 149-156, 2001). These promoters confer high levels of expression in most animal tissues, and are generally not dependent on the particular encoded proteins to be expressed. As described below, the CMV promoter was found to be effective in driving expression of genes encoding IGF-1 fusion proteins in transduced human RPE cells. Other promoters that may be useful in the invention can include the Rous sarcoma virus promoter, adenovirus major late promoter (MLP), herpes simplex virus (HSV) promoter, HIV long terminal repeat (LTR) promoter, mouse mammary tumor virus LTR promoter, β-actin promoter (Genbank # K00790), or the murine metallothionein promoter (Stratagene San Diego Calif.). Synthetic promoters, hybrid promoters, and the like are also useful in the invention and are known in the art.

Animal expression vectors may also include RNA processing signals such as introns, which have been shown to increase gene expression (Yu et al., 2002, 81: 155-163; Gough et al., 2001, Immunology 103: 351-361). The location of the RNA splice sequences can influence the level of transgene expression in animals. In view of this fact, an intron may be positioned upstream or downstream of a growth factor protein-encoding sequence in the transgene to modulate levels of gene expression. Expression vectors within the invention may also include regulatory control regions which are generally present in the 3′ regions of animal genes. See, for example, Jacobson et al., 1996, Annu Rev Biochem 65:693-739; and Rajagopalan et al., 1997, Prog Nucl Acid Res Mol Biol 56:257-286. For example, a 3′ terminator region may be included in the expression vector to increase stability of the MRNA.

Animal expression vectors within the invention preferably contain a selectable marker gene used to identify the cells that have become transformed. Suitable selectable marker genes for animal systems include genes encoding enzymes that produce antibiotic resistance (for example, those conferring resistance to hygromycin, kanamycin, bleomycin, neomycin G418, and streptomycin).

Vectors further encompassed by the invention can also include those that result in the production of growth factor fusion proteins, such as growth factors fused with a detectable label or “reporter protein,” for example, green fluorescent protein (GFP), luciferase or β-galactosidase. Detectable labels are useful, for example, to distinguish transgenic growth factors from endogenous forms of the same factor. Also of utility are growth factors fused with an “epitope tag,” for instance, His₆. Epitope tags are useful for isolating the tagged fusion protein, for example, by chromatography on a nickel column, for identifying the fusion protein with an epitope tag-specific antibody, and for enabling differentiation of native and fusion protein growth factors by virtue of their differing molecular weights. In embodiments of the invention described below, backbone plasmids pEGFP-C1 and pCDNA3.1His C, respectively, were used to construct vectors directing expression of human IFG-1 fusion proteins linked to GFP, and to a His₆-epitope tag.

### Growth Factor-Encoding Nucleic Acids

The present invention utilizes expression vectors incorporating growth factor-encoding nucleic acids to direct synthesis of growth factor proteins in transduced cells. Growth factor-encoding nucleic acid molecules of use in the invention can include the DNA sequence encoding the polypeptide sequence of any growth factor known to impart a neurotrophic or neuroprotective effect on cells of the retina, including at least one of IGF-1, bFGF, aFGF, CNTF, PDGF, and BDNF. Methods for amplification of full-length and partial coding sequences of known genes, for example, by RT-PCR using specific primers based on published sequences, are well known to those of skill in the art, and are further described below. As an example, primers having the nucleic acid sequences of SEQ ID NOs: 1 and 2 can be utilized to amplify the CDNA sequence of human IGF-1 (NCBI GenBank gi#14758742), flanked by additional restriction enzyme recognition sequences useful for ligating the IGF-1 cDNAs into expression vectors.

Nucleic acid molecules utilized in the present invention may be in the form of RNA or in the form of DNA (for example, cDNA, genomic DNA, or synthetic DNA). The DNA may be double-stranded or single-stranded, and if single-stranded, may be the coding (sense) strand or non-coding (anti-sense) strand. The coding sequence which encodes a native growth factor protein may be identical to a published polynucleotide sequence for any known growth factor, for example, IGF-1, bFGF, aFGF, CNTF, PDGF, and BDNF. It may also be a different coding sequence which, as a result of the redundancy or degeneracy of the genetic code, encodes the same polypeptide as one of these polynucleotides.

Other nucleic acid molecules within the invention are variants of a native growth factor gene such as those that encode fragments (for example, post-translationally processed forms of), analogs and derivatives of a native growth factor protein. Such variants may be, for example, a naturally occurring allelic variant of a native growth factor gene, a homolog of a native factor gene, or a non-naturally occurring variant of a native growth factor gene. These variants can have a nucleotide sequence that differs from a native growth factor gene in one or more bases. For example, the nucleotide sequence of such variants can feature a deletion, addition, or substitution of one or more nucleotides of a native growth factor gene. Nucleic acid insertions are preferably of about 1 to 10 contiguous nucleotides, and deletions are preferably of about 1 to 30 contiguous nucleotides.

In other applications, variant growth factor proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, for example, serine or threonine, for (or by) a hydrophobic residue, for example, leucine, isoleucine, phenylalanine, valine or alanine; (b) a cysteine or proline for (or by) any other residue; (c) a residue having an electropositive side chain, for example, lysine, arginine, or histadine, for (or by) an electronegative residue, for example, glutamine or aspartine; or (d) a residue having a bulky side chain, for example, phenylalanine, for (or by) one not having a bulky side chain, for example, glycine.

Naturally occurring allelic variants of a native growth factor gene within the invention are nucleic acids isolated from human or murine tissue that have at least 75% (for example, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with a native growth factor gene, and encode polypeptides having structural similarity to a native growth factor protein. Homologs of a native growth factor gene within the invention are nucleic acids isolated from other species that have at least 75% (for example, 76%, 77%, 78%, 79%, 80%, 81%, 82%, 83%, 84%, 85%, identity with a native growth factor gene, and encode polypeptides having structural similarity to native growth factor protein. Public and/or proprietary nucleic acid databases can be searched in an attempt to identify other nucleic acid molecules having a high percent (for example, 70, 80, 90% or more) sequence identity with a native growth factor gene.

Non-naturally occurring growth factor gene variants are nucleic acids that do not occur in nature (for example, are made by the hand of man), have at least 75% (for example, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with a native growth factor gene, and encode polypeptides having structural similarity to a native growth factor protein. Examples of non-naturally occurring growth factor gene variants are those that encode a fragment of a growth factor protein, those that hybridize to a native growth factor gene or a complement of a native growth factor gene under stringent conditions, those that share at least 65% (for example, 66%, 67%, 68%, 69%, 70%, 71%, 72%, 73%, 74%, 75%, 76%, 77%, 78%, 79%, 80%, 81%, 82%, 99%) sequence identity with a native growth factor gene or a complement of a native growth factor gene, and those that encode a growth factor fusion protein.

Nucleic acids encoding fragments of a native growth factor protein within the invention are those that encode, for example, 2, 5, 10, 25, 30, 40, 50, 60, 70, 80, 90, 100, or more amino acid residues of a native growth factor protein. Shorter oligonucleotides (for example, those of 6, 7, 8, 9, 10, 11, 12, 13, 14, 15, 16, 17, 18, 19, 20, 30, 50, 100, base pairs in length) that encode or hybridize with nucleic acids that encode fragments of a native growth factor protein can be used as probes, primers, or antisense molecules. Longer polynucleotides (for example, those of 125, 150, 175, 200, 225, 250, 275, 300, or more base pairs) that encode or hybridize with nucleic acids that encode fragments of a native growth factor protein can also be used in various aspects of the invention. Nucleic acids encoding fragments of a native growth factor protein can be made by enzymatic digestion (for example, using a restriction enzyme) or chemical degradation of the full length native growth factor gene or variant thereof.

Nucleic acid molecules encoding growth factor fusion proteins are also within the invention. Such nucleic acids can be made by preparing a construct (for example, an expression vector) that expresses a growth factor fusion protein when introduced into a suitable host. For example, such a construct can be made by ligating a first polynucleotide encoding a growth factor protein fused in frame with a second polynucleotide encoding another protein (for example, a detectable label or affinity tag) such that expression of the construct in a suitable expression system yields a fusion protein. In examples described herein, methods are provided for using isolated nucleic acids encoding growth factors (for example, IGF-1) ligated into expression vectors designed to produce IGF-1 fusion proteins containing either a reporter protein, (for example a fluorescent marker such as GFP), or an epitope tag (for example, His₆).

The nucleic acids of the vectors of invention can be modified at the base moiety, sugar moiety, or phosphate backbone, for example, to improve stability of the molecule, hybridization, etc. The nucleic acids within the invention may additionally include other appended groups such as peptides (for example, for targeting host cell receptors in vivo), or agents facilitating transport across the cell membrane.

### Kits

In another aspect, the invention provides kits useful for gene transduction of a cell. The kits can include at least one expression vector containing a nucleic acid encoding a human growth factor protein fused with GFP, or a nucleic acid encoding a human growth factor protein fused with an epitope tag, and instructions for use. In one embodiment, the kit can contain at least one expression vector that directs expression of a human IGF-1 protein linked to a His₆ epitope tag. In another embodiment, the kit can include at least one expression vector that directs expression of a human IGF-1 fusion protein linked to a reporter protein such as GFP.

### Cells Transduced with Growth Factor Vectors

In yet another aspect, the invention provides retinal cells, for example, RPE cells, transduced with growth factor vectors, and methods of using such cells. One embodiment of the method of increasing synthesis or secretion of a growth factor by a RPE cell includes the step of transducing RPE cells with a vector that contains a growth factor-encoding nucleic acid. Several standard methods are known for “transducing,” or “transfecting,” cells, that is, introducing recombinant genetic material into the host cell or animal for the generation of a transgenic cell or animal. Examples of such methods include (1) particle delivery systems (see for example, Novakovic S et al., 1999 J Exp Clin Cancer Res 18:531-536; Tanigawa et al., 2000, Cancer Immunol Immunother 48:635-643); (2) microinjection protocols (see, for example, Krisher et al., 1994, Transgenic Res 3:226-231; Robinett C C and Dunaway M,1999, In, Methods: A Companion to Methods in Enzymology 17:151-160; or Pinkert C A and Trounce I A ,2002, Methods 26:348-57); (3) polyethylene glycol (PEG) procedures (see for example, Meyer O et al., 1998, J Biol Chem 273:15621-15627; or Park et al., 2002, Bioconj Chem 13:232-239); (4) electroporation protocols (see for example, Dev S B and Hofinann G A, 1996, In: Lynch P T and Davey M R, eds., Electrical Manipulation of Cells, Chapman & Hall, New York, 185-199 ); and (5) liposome-mediated DNA uptake (see, for example, Hofland H E J and Sullivan S M, 1997, J Liposome Res 7:187-205; or Hui S W et al., 1996, Biophys J 71:590-599).

For transducing RPE cells, any suitable method can be used. A particularly attractive method is liposome-mediated DNA uptake. As shown below, this method is highly effective for introduction of plasmid DNA into RPE cells derived from aged humans. The efficiency of transduction is thought to relate to the endogenous phagocytic capability of RPE cells (Chaum E et al., 1999, J Cell Biochem 76:153-160; Chaum E, 2001, Cell Biochem 83:671-677). Similarly, it is contemplated that the vectors of the invention could be used to transduce RPE cells in situ, for example, by providing the vectors to the RPE cells, via delivery into the subretinal space. Methods for achieving ocular delivery of vectors to retinal cells are further discussed below.

Studies disclosed herein indicate that introduction and expression of growth factor genes using techniques of gene transfer may effect beneficial alterations in the proliferative potential of RPE cells. It is contemplated that increased expression of growth factors like IGF-1 in transduced cells in vivo may create “cytokine factories” within the RPE cell population in situ. This reservoir of transduced cells would be expected to increase the relative concentration of effector cytokines through diffusion into the subretinal space, and possibly into the choroid underlying the RPE. Increased growth factor presence, especially in the aging eye, may enhance RPE and retinal cell survival and function.

### Expressed Growth Factor Proteins

The present invention further provides purified growth factor proteins, including fusion proteins, the expression of which is directed in a cell transduced by a growth factor-encoding nucleic acid incorporated into an expression vector. As described above, growth factor-encoding nucleic acids can be used to direct expression of growth factor proteins in transduced cells, for example, RPE cells. Accordingly, forms of the resultant expressed growth factor protein can vary according to the nucleic acid sequence that encodes the protein, and can include a purified native growth factor protein that has the amino acid sequence of a native growth factor protein, for example that of human IGF-1, bFGF, aFGF, CNTF, PDGF, and BDNF. Variants of native growth factor proteins such as fragments, analogs and derivatives of a native growth factor protein are also within the invention. Such variants can include, for example, a polypeptide encoded by a naturally occurring allelic variant of a native growth factor gene, a polypeptide encoded by a homolog of a native growth factor gene, and a polypeptide encoded by a non-naturally occurring variant of native growth factor gene.

Growth factor protein variants have a peptide sequence that differs from a native growth factor protein in one or more amino acids. The peptide sequence of such variants can feature a deletion, addition, or substitution of one or more amino acids of a native growth factor polypeptide. Amino acid insertions are preferably of about 1 to 4 contiguous amino acids, and deletions are preferably of about 1 to 10 contiguous amino acids. In some applications, variant growth factor proteins substantially maintain a native growth factor protein functional activity. For other applications, variant growth factor proteins lack or feature a significant reduction in a growth factor protein functional activity. Where it is desired to retain a functional activity of a native growth factor protein, preferred growth factor protein variants can be made by expressing nucleic acid molecules that feature silent or conservative changes. Variant growth factor proteins with substantial changes in functional activity can be made by expressing nucleic acid molecules that feature less than conservative changes.

Growth factor protein fragments corresponding to one or more particular motifs and/or domains or to arbitrary sizes, for example, at least 5, 10, 25, 30, 40, 50, 60, 70, 75, 80, 90, and 100 amino acids in length are within the scope of the present invention. Isolated peptidyl portions of growth factor proteins, for example, IGF-1, bFGF, aFGF, CNTF, PDGF, and BDNF, can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a growth factor protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a native growth factor protein.

Another aspect of the present invention concerns recombinant forms of growth factor proteins. Recombinant polypeptides preferred by the present invention are encoded by a nucleic acid that has at least 85% sequence identity (for example, 85, 86, 87, 88, 89, 90, 91, 92, 93, 94, 95, 96, 97, 98, 99, 100%) with the nucleic acid sequence of one of the growth factors including IGF-1, bFGF, aFGF, CNTF, PDGF, and BDNF. In a preferred embodiment, variant growth factor proteins have one or more functional activities of a native factor protein.

Growth factor protein variants can be generated through various techniques known in the art. For example, growth factor protein variants can be made by mutagenesis, such as by introducing discrete point mutation(s), or by truncation. Mutation can give rise to a growth factor protein variant having substantially the same activity, or merely a subset of the functional activities of a native growth factor protein. Alternatively, antagonistic forms of the protein can be generated which are able to inhibit the function of the naturally occurring form of the protein, such as by competitively binding to another molecule that interacts with a growth factor protein. In addition, agonistic forms of the protein may be generated that constitutively express one or more growth factor functional activities. Other variants of growth factor proteins that can be generated include those that are resistant to proteolytic cleavage, as for example, due to mutations which alter protease target sequences. Whether a change in the amino acid sequence of a peptide results in a growth factor protein variant having one or more functional activities of a native growth factor protein can be readily determined by testing the variant for a native growth factor protein functional activity. For example, as described below, a growth factor fusion protein (such as His₆-tagged hIGF-1, or hIGF-1:GFP) can be tested for ability to stimulate proliferation of RPE cells, to promote recruitment into the cell cycle, or to promote survival of photoreceptors or RPE cells in an animal model of retinal degeneration.

The present invention further pertains to methods of producing the subject growth factor proteins. For example, a host cell transfected with a vector directing expression of a nucleic acid sequence encoding the subject growth factor polypeptides can be cultured under appropriate conditions to allow expression of the polypeptide to occur. The cells may be harvested, lysed, and the protein isolated. A recombinant growth factor protein can be isolated from host cells using techniques known in the art for purifying proteins including ion-exchange chromatography, gel filtration chromatography, ultrafiltration, gel electrophoresis (for example SDS-PAGE), and immunoaffinity purification with antibodies specific for such protein.

A growth factor protein expressed in a cell can be isolated, for example, using immunoaffinity chromatography as follows. A growth factor-specific antibody (for example, an antibody that specifically binds to human IFG-1, bFGF, aFGF, CNTF, PDGF, or BDNF, as appropriate, can be immobilized on a column chromatography matrix, and the matrix can be used for immunoaffinity chromatography to purify the particular growth factor protein from cell lysates by standard methods (see, for example, Ausubel et al., supra). Following immunoaffinity chromatography, the recombinant protein can be further purified by other standard techniques, such as high performance liquid chromatography.

In embodiments in which a growth factor protein is expressed as a fusion protein containing an epitope tag (for example, His₆), the tag on the protein can be used to facilitates its purification, for example, on a nickel column. An example of this method is further described below, in which a His₆-tagged human IGF-1 fusion protein was purified following transduction and expression of the protein in cultured human RPE cells.

### Detecting Expressed Growth Factor Nucleotides and Proteins

The invention provides a method of increasing synthesis or secretion of a growth factor by a RPE cell by transducing the cell with a growth factor-expressing vector. Numerous methods are available for detecting the presence in a cell of an expressed nucleic acid or protein, such as those of growth factors, or detecting protein secreted extracellularly by the cell, and for measuring the levels of growth factor nucleic acid and protein in biological samples.

An exemplary method for detecting the presence or absence of a growth factor in a biological sample involves obtaining a biological sample from a test subject (for example, a rodent or a human patient, or a culture of cells prepared from such subjects), contacting the biological sample with a compound or an agent capable of detecting a growth factor protein or a nucleic acid encoding a growth factor protein (for example, mRNA or genomic DNA), and analyzing binding of the compound or agent to the sample after washing. Those samples having specifically bound the compound or agent are those that express the growth factor protein or nucleic acid encoding the growth factor protein.

A preferred agent for detecting a nucleic acid encoding a growth factor protein is a labeled nucleic acid probe or primer capable of hybridizing (for example, under stringent hybridization conditions) to the nucleic acid encoding a growth factor protein. The nucleic acid probe or primer can be, for example, all or a portion of a native growth factor gene itself (for example, human IFG-1, bFGF, aFGF, CNTF, PDGF, or BDNF), or all or a portion of a complement of a native growth factor gene. Similarly, the probe or primer can also be all or a portion of a growth factor gene variant, or all or a portion of a complement of a growth factor gene variant. For instance, oligonucleotides at least 15, 30, 50, 75, 100, 125, 150, 175, 200, 225, or 250 nucleotides in length that specifically hybridize under stringent conditions to a native growth factor gene or to a complement of a native growth factor gene can be used as probes or primers within the invention.

Examples of specific embodiments of nucleic acid primers useful in the invention are those having DNA sequences listed as SEQ ID NOs: 1-7. All of these sequences are related to detecting the human IFG-1 gene. As further described in the examples, the primer pair indicated as SEQ ID NOs: 1 and 2 can be used to amplify a human IGF-1 cDNA, flanked with restriction enzyme sites useful for ligation of the cDNA into plasmid expression vectors. Primers identified as SEQ ID NOs: 3-7 are useful for identifying and amplifying selected portions of the human IGF-1 CDNA, and for distinguishing between the native growth factor (IGF-1), and a transgenic form of an IGF-1 fusion protein (His-tagged IGF-1, or IGF1:GFP).

Techniques for detection of genomic DNA encoding a growth factor protein using probes include Southern hybridization. Techniques using probes for detection of mRNAs encoding growth factor proteins include Northern hybridization and in situ hybridization.

For detecting a growth factor protein, a preferred agent is an antibody capable of binding to a growth factor protein, preferably an antibody with a detectable label. Such antibodies can be polyclonal, or more preferably, monoclonal. An intact antibody, or a fragment thereof (for example, Fab or F(ab′)₂) can be used. Techniques for detecting a growth factor protein include enzyme linked immunosorbent assay (ELISA), Western blotting, immunoprecipitation and immunofluorescence. As an example, as described below, an antibody directed against human IGF-1 can be used in a western blot analysis to detect both synthesis of a transgenic human IGF-1 fusion protein by transfected human RPE cells, and secretion of the fusion protein into the culture medium.

Techniques for detection of a growth factor protein in vivo can include introducing a labelled anti-growth factor specific antibody into a biological sample or test subject. For example, the antibody can be labeled with a radioactive marker whose presence and location in a biological sample or test subject can be detected by standard imaging techniques. Sites of localization of growth factors can be also be detected by immunostaining of sections of biological tissues from subjects using growth factor-specific antibodies, including those recognizing tagged fusion proteins such as His₆-tagged fusion proteins.

### Methods of Treatment

In yet a further aspect, the invention provides a method of treating dysfunction or injury of a RPE cell. The method relates to transducing a RPE cell with an expression vector that expresses a growth factor protein in an amount sufficient to improve or cure the dysfunction or injury. The method can be used to treat any dysfunction or injury of the RPE thought to benefit from the neurotrophic effects of enhanced expression of one or more growth factors. The causes of such dysfunctions affecting the RPE cell are myriad, and can include cancer, viral infection such as CMV retinitis, systemic diseases that affect the eye, such as diabetes and gyrate atrophy, hereditary forms of RPE dystrophy (for example Stargardt's disease/fundus flavimaculatus, Best disease/vitelliform dystrophy, congenital diffuse drusen/Doyne's honeycomb dystrophy, pattern dystrophies, Sorsby's macular dystrophy, choroideremia, and idiopathic bulls-eye maculopathies), as well as AMD. Secondary RPE degeneration in retinitis pigmentosa conditions may also be a target of therapy. Non-genetic disease targets can include toxic maculopathies, for example, drug-induced maculopathies such as plaquenil toxicity.

The method may also have application in conditions affecting the RPE that manifest as inability to divide, or commitment along the pathway to programmed cell death (apoptosis). The latter conditions may be especially amenable to treatment of the dysfunctional RPE with a transducing vector that directs expression of enhanced levels of IGF-1, since this growth factor has been demonstrated herein to increase the proliferative potential of transduced RPE cells from aged humans, and has been shown to have anti-apoptotic properties.

The method may also be useful for treatment of injured RPE cells. RPE injuries can result from various sources, including ocular trauma (for example, leading to macular commotio), photic toxicity, laser-induced injury, surgical procedures, or retinal detachment.

The invention further provides a method of treating a retinal disease or condition in a subject. This method may be performed by introducing into the subject a composition including an expression vector containing a nucleic acid encoding a growth factor in an amount sufficient to ameliorate or cure the disease or condition. The introduced vectors can produce one or more growth factors, including IFG-1, bFGF, aFGF, CNTF, PDGF, or BDNF. In preferred embodiments, the growth factor is human IGF-1.

Suitable subjects for use in the invention can be any animal. For example, the subject can be an animal such as mammal like a dog, cat, horse, cow, pig, sheep, goat, chicken, primate, rat, or mouse. Preferred are subjects suspected of having, or at risk for developing, a retinal disease or disorder, for example, a person suspected of having, or at risk for developing, any form of hereditary retinal degeneration, retinitis pigmentosa, diabetic retinopathy, hereditary RPE dystrophy, or AMD, based on clinical findings or other diagnostic test results. Other suitable subjects include patients at risk for drug-induced RPE injury, or patients with a history of ocular trauma or retinal detachment.

Examples of hereditary RPE dystrophies that may be appropriate disease targets for the disclosed methods and compositions include such conditions as Stargardts/fundus flavimaculatus, Best Disease/Vitelliforn dystrophy, congenital diffuse drusen (Doyne's honeycomb dystrophy), pattern dystrophies, Sorsby's macular dystrophy, choroideremia, gyrate atrophy and idiopathic bulls-eye maculopathies. Secondary RPE degeneration in patients with retinitis pigmentosa conditions may also be targets of therapy. Non-genetic disease targets can further include toxic maculopathy (drug-induced, such as plaquenil toxicity), trauma, including photic toxicity, laser-induced injury, and macular commotio from blunt ocular injury.

The compositions of the invention can be administered to animals or humans by any conventional technique. Such administration might be parenteral (for example, intravenous, subcutaneous, intramuscular, or intraperitoneal introduction). Preferably, the compositions may be administered directly to the target site (for example, to the eye, or to a compartment of the eye, such as the vitreous or subretinal space), for example, by intraocular injection.

An effective amount of growth factor expression vector sufficient for ameliorating or curing the retinal disease or condition can be determined by established procedures for evaluation of outcomes of gene therapy procedures in the eye. For example, the effects of gene transfer of growth factor vectors into retinal cells can be first determined in animal models of retinal degenerative conditions. A wide variety of such models is in existence (both naturally occurring mutants and transgenic models), in such species as rats, mice, dogs, cats, chickens, monkeys and pigs, and available for analysis of many types of retinal degenerative conditions affecting photoreceptors and RPE cells.

In general, determination of an effective amount of the composition is made in the subject before and after administration of the compositions, using standard methods known in the art, such as measurements of: 1) rates of photoreceptor degeneration; 2) rates of RPE cell survival; 3) rates of proliferation of the RPE; 4) rates of apoptosis; 5) levels of RPE phagocytic ability; 6) levels of growth factor mRNA and protein expression, among others, as well as verification of lack of undesired effects (for example, development of abnormal blood vessels).

Determination of an effective amount of growth factor vector for gene transfer to a human subject is guided by results from animal studies. Outcomes in human patients are monitored in controlled studies using standardized clinical protocols, and appropriate measurement techniques (such as funduscopy, fluorescein angiography, indocyanine green angiography, electroretinography, visual evoked potentials, visual field testing, contrast sensitivity, Snellen acuity, and the like) known to retinal specialists experienced in the management of patients with such disorders.

### EXAMPLES

The following examples serve to illustrate the invention without limiting it thereby. It will be understood that variations and modifications can be made without departing from the spirit and scope of the invention.

### Example 1

### RPE Cell Culture

Human RPE cells were isolated from cadaver eyes provided by the Mid-South Eye Bank (Memphis) using methods as described (Chaum E, J Cell Biochem 2001, 83:671-677). RPE cells were maintained in Dulbecco's modified Eagle media (DMEM) supplemented with fetal calf serum (FCS) (Atlanta Biologicals, Norcross, Ga.) plus L-glutamine, penicillin, and streptomycin in an atmosphere of humidified 95% air and 5% CO₂ at 37° C. The concentration of FCS was specific to the methods of each experiment as described. The cells exhibited cuboidal monolayer growth characteristics in the human eye in situ, but became somewhat less hexagonal with serial subculture. Cells were transfected during the third to fourth subculture in vitro. The clones derived from the transfections were analyzed from passages 4 to 8.

### Example 2

### IGF-1 Fusion Gene Vectors

Amplification of IGF-1 cDNA. Plasmid pAX-IGF-1 (gift from Drs. Renato Baserga and Gary Stein) contains a cDNA copy of the human IGF-1 gene (hIGF-1) (National Center for Biotechnology Information [NCBI] GenBank gi#14758742). The plasmid was used as a DNA template in PCR amplification reactions performed for vector construction. Forward and reverse primers encoding unique restriction enzyme sites linked to short flanking sequences from the 5′ ends of each strand of the hIGF-1 cDNA were synthesized. Primer 1 (5′-ATA AGA TCT GGA TCC ATG CAC ACC ATG TCC-3′; Tm=65.4) (SEQ ID NO: 1) is a forward primer identical to the nucleotide sequence from positions 81 to 92 of the hIGF-1 cDNA, with additional Bg1 II and Bam H1 restriction enzyme cleavage sites added in the 5′ region. The reverse primer, primer 2 (3′-T CCT TTG TTC TTG ATG TCC TAC ATC CTT AAG AGA TCT -5′, Tm=63.0) (SEQ ID NO: 2) is complementary to positions 442 to 473 of the hIGF-1 cDNA, with an Eco R1 restriction enzyme cleavage site added in the 5′ region. The modified 405-bp hIGF-1 cDNA coding sequence, in which the cDNA was flanked by the new restriction enzyme digest sites, was then PCR-amplified, using pAX-IGF-1 as a template. The resulting amplification product was cut from the gel and purified using a QIAEX II gel extraction kit (Quiagen), for subsequent insertion into the pEGF:IGF-1 and pcDNA:IGF-1 vectors, as described below.

Construction of pEGFP:IGF-1 fusion gene vector. A vector expressing a fusion protein containing human IGF-1 linked to a fluorescent marker (i.e., green fluorescent protein, GFP) was constructed as follows. A pEGFP-C1 plasmid backbone (Clontech, Palo Alto, Calif.) was used to construct a pEGFP:IGF-1 fusion gene. The pEGFP-C1 plasmid backbone contains a GFP gene, as well as a selectable marker gene for neomycin resistance (neo^{r}) and antibiotic resistance genes, which permit plasmid amplification in E. coli HB101using standard transformation methods.

The amplified human IGF-1 PCR product, prepared as described above, was inserted downstream of the CMV-promoted GFP gene into the multiple cloning sequence (MCS) of the pEGFP-C1 plasmid. Briefly, the amplified IGF-1 PCR product was double digested with Bg1 II and Eco R1 for insertion into the plasmid vector. The pEGFP-C1 plasmid was sequentially digested with Bg1II, followed by Eco R1, for 2 hours at 37° C. in the appropriate digest buffer. All digested DNA was precipitated in ethanol following restriction enzyme digestion and dissolved in Tris/EDTA (10:1) and subsequently ligated at 16° C. overnight, to produce the pEGFP:IGF-1 fusion gene.

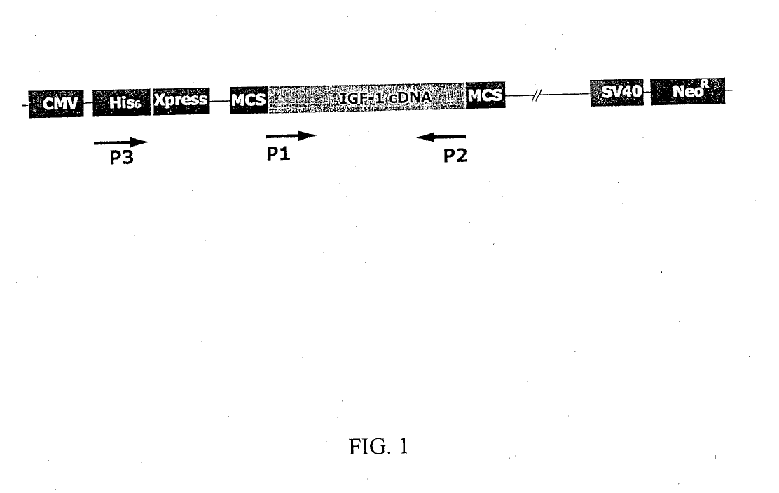

Construction of pcDNA:IGF-1 His₆-tagged fusion gene vector. A vector expressing a fusion protein containing human IGF-1 linked to a 6 amino acid (His₆) tag was constructed as follows. A pcDNA3.1/HisC plasmid backbone (Invitrogen, Carlsbad, Calif.) was used to construct the pcDNA:IGF-1 fusion gene. As depicted in FIG. 1, the pcDNA3.1/HisC plasmid backbone contains His₆ and Xpress epitope sequences downstream of a CMV promoter, as well as a selectable marker gene for neomycin resistance (neo^{r}). The plasmid and the amplified human IGF-1 PCR product were subjected to appropriate restriction enzyme digestion as above, followed by insertion of the IGF-1 cDNA into the MCS downstream of the His₆ and Xpress epitope sequences. The epitope sequence, linker region, and IGF-1 cDNA were sequenced. No mutations were generated from the plasmid construction, and the IGF-1 gene was confirmed to remain in frame.

Ligated plasmids were used to transform E. coli HB101. Antibiotic-resistant bacterial colonies were screened for the presence of recombinant plasmids (i.e., pEGFP:IGF-1 and pcDNA:IGF-1). Colonies containing recombinant plasmid of the appropriate molecular weight were amplified and the structure of the plasmid was confirmed by restriction enzyme digestion analysis.

### Example 3

### Transfection and Cloning of RPE Cells

Human RPE cells were transfected in vitro using the dendrimer Superfect as previously described (Blakesley V A et al., In Rosenfeld, R and Roberts, C eds., Contemporary Endocrinology: The IGF-1 System, Totowa, N.J., Humana Press; 1999:143-163). Cells were fed with media containing 16% FCS and 500 μg/mL of G418 (Sigma, Saint Louis, Mo.) 48 hours after transfection to select for clones that expressed the neo^{r} transgene. G418-resistant clones were isolated during the ensuing 2 to 4 weeks using cloning cylinders, and propagated in media containing 125 μg/mL of G418. Clones were selected that demonstrated evidence of stable transfection of an epitope-tagged human IGF-1 cDNA fusion gene. Transduced RPE cells were cloned using a selectable antibiotic resistance marker. Transfected clones were screened for fusion gene transcription using reverse transcriptase-polymerase chain reaction (RT-PCR) methods as described below. A G418-cloned RPE cell line expressing undetectable levels of GFP fusion transgene was used as an additional control for cell proliferation studies.

### Example 4

### RT-PCR and Real-Time Quantitative RT-PCR

Total RNA was isolated from RPE cells with TRI Reagent (Sigma), using the protocol recommended by the manufacturer, and stored at −80° C. First strand cDNA synthesis was performed with 1 μg of total RNA as a template with the Reverse Transcription System (Promega, Madsion, Wis.) using the manufacturer's recommended protocol. One-fifth of the first strand cDNA was transferred to a vial containing 80 μl of 1× PCR Master Mix (Promega) and forward and reverse primers as described below. RT-PCR amplification was performed in a Mastercycler (Eppendorf Scientific, Westbury, N.Y.). Samples were treated for 5 minutes at 94° C., then 30 cycles of amplification were performed as follows: 45 seconds at 94° C., followed by 45 seconds at the annealing temperature, and 90 seconds at 72° C., with final extension at 72° C. for 10 minutes.

Referring to FIG. 1, forward primer 1 (5′-ATG CAC ACC ATG TCC TC-3′; SEQ ID NO: 3) and reverse primer 2 (3′-CTT TGT TCT TGA TGT CCT AC-5′; SEQ ID NO: 4) are embedded within the IGF-1 cDNA. This primer set amplifies a 393-bp oligonucleotide from both the endogenous cellular IGF-1 and transfected IGF-1 fusion gene transcripts. By contrast, forward primer 3 (5′-ATG GGG GGT TCT CAT CAT-3′; SEQ ID NO: 5) and reverse primer 2 (above) amplify a 487-bp pcDNA:IGF-1-specific fusion gene transcript. β-actin transcripts were amplified from each RNA sample using β-actin-specific primers as an internal control (Promega). The RT-PCR amplification products were subjected to SDS PAGE gel electrophoresis, stained with ethidium bromide, and imaged using the Typhoon 8600 Variable Mode Imager (Molecular Dynamics, Sunnyvale, Calif.).

Real-time quantitative RT-PCR (qRT-PCR) was performed using the SYBR Green method. Real-time RT-PCR primers for IGF-1 were designed using the ABI PrimerExpress 1.5 software (Applied Biosystems, Foster City, Calif.) (forward, 5′-TTT CAA CAA GCC CAC AGG GT-3′(SEQ ID NO: 6); reverse, 3′-G GAG TCT GTC CGT AGC ACC-5′(SEQ ID NO: 7). Optimization of SYBR Green PCR amplification using different IGF-1 primer pair ratios was performed according to the manufacturer's recommendations. To amplify the IGF-1 transcript, 5 μl of the reverse transcription product, and the optimized ratio of the primers (1:1) were added to 1× SYBR Green PCR Master Mix (Applied Biosystems, Werrington, UK). Real-time qRT-PCR was performed using the ABI PRISM 7700 Sequence Detection System (Applied Biosystems). After 10 minutes at 95° C., 40 cycles of 15 seconds at 95° C. followed by 1 minute at 60° C. was performed. The cycle at which each sample reached the critical amplification threshold (C_{T} value) was determined. β-actin transcript amplification was used as an internal control.

A quantitative IGF-1 calibration curve was established using serial dilutions of IGF-1 cDNA at concentrations ranging from 1 to 0.0001 ng/μl. The concentration of IGF-1-specific transcript present in the RNA samples was quantified by comparing the C_{T} values of the RPE samples with known C_{T} values from the calibration curve.

### Example 5

### Western Blot Analysis

RPE tissue culture monolayers were lysed in situ (1% w/v, Triton X-100, 50 mM Tris-Cl [pH 7.4], 300 mM NaCI, 5 mM EDTA) with additional proteinase inhibitors (20 μg/mL aprotinin, 20 μg/mL leupeptin, 20 μg/mL pepstatin, 1 mM phenylmethylsulfonyl fluoride and 10 mM iodoacetamide). The protein concentrations of the cell lysates were determined by spectrophotometry using the Bradford method. Thirty micrograms of whole-cell protein lysate (in 1× Tris-glycine-SDS sample buffer with 100 mM dithiothreitol (DTT) from each clone was separated by electrophoresis in 16% Novex Tris-tricine gels (Invitrogen). The gels were semi-dry transferred to nitrocellulose membranes (Protran, Schleicher & Schuell Inc., Keene, N.H.) for blotting.

The membranes were blocked with SuperBlocker buffer (Pierce, Rockford, Ill.) in Tris-buffered saline with 0.2% Tween 20, pH 7.6 (TBS-T) for 2 hours at 4° C. Membranes were incubated with primary antibodies of goat anti-hIGF-1 (Sigma) at a dilution of 1:6000 in TBS-T at 4° C. overnight. The membranes were reblocked with SuperBlocker for 1 hour at room temperature (RT) prior to incubation with secondary antibodies of Biotin-SP-conjugated AffiPure F(ab′)2 fragment rabbit anti-goat IgG (Jackson Laboratory, Bar Harbor, Me.) at a dilution of 1:80,000 in TBS-T for 1 hour at RT. After washing with TBS-T, the membranes were incubated with peroxidase-conjugated streptavidin (Jackson Laboratory) at a dilution of 1:40,000 in TBS-T for 1 hour at RT. Membranes were incubated with ECL+plus (Amersham Pharmacia, Piscataway, N.J.) for 5 minutes at room temperature, and the chemiluminescent signals were captured on Hyperfilm. Densitometry measurements were performed using a Typhoon 8600 imager.

The transgenic IGF-1 protein carrying the His₆ tag was isolated by nickel column chromatography (Ni-TED; Active Motif, Carlsbad, Calif.). This method was found to be effective for selective binding and elution of the His₆-tagged protein. Western blotting was performed as described above on the protein eluted from the column to confirm the presence of the His₆-tag on the IGF-1 fusion protein.

### Example 6

### Analyses of Cellular Proliferation

RPE cell proliferation assays. Proliferation assays were performed to compare the growth rates of IGF-1-transduced clones with controls. The proliferation of RPE cells was assessed by a quantitative CCK-8 assay, using the protocol recommended by the manufacturer (Cell Counting Kit-8, Dojindo, Gaithersburg, Md.). Briefly, the CCK-8 method is an optical density (O.D.) colorimetric assay that quantifies the number of viable cells per well based upon the activity of cellular dehydrogenases. Clones were plated in tissue culture plates at a density of 5×10³ cells per well and grown in DMEM with 0.5% FCS. The number of cells per well was quantified by O.D. at 1, 3, and 6 days following plating. Replicate growth curves (n=4) were plotted for the each of the IGF-1-transduced clones and compared to controls grown under identical culture conditions. The cells were not grown in serum-free conditions because IGF-1 alone is not sufficient to induce quiescent cells to cycle.

Flow cytometric studies. Assays were performed on synchronized RPE cells. Replicate cultures (n=4) of 5×10⁵ cells were plated in tissue culture wells and subjected to 48 hours of serum starvation, after which time the cells were refed with media containing 0.5% FCS. Cell monolayers were harvested by trypsinization 27 hours after release from serum starvation. The cells were washed with ice-cold buffered saline containing 1% bovine serum albumin (BSA buffer) and fixed in 70% ethanol (−20° C.). Fixed cells were resuspended in BSA buffer containing 100 μg/mL RNAse A (Sigma) and 5 μg/mL propidium iodide (PI) (in sodium citrate buffer containing Triton X-100) at 37° C. for 15 minutes in the dark. The PI fluorescence of the cell suspensions was quantified by flow cytometry using a Beckton Dickinson (Franklin Lakes, N.J.) flow cytometer. Data histograms of cell ploidy were analyzed using the ModFit LT software program (Verity Software, Topsham, Me.).

### Example 7

### Morphology of Transduced Clones

No morphologic changes apparent at the light microscopic level were induced by expression of the neo^{r} transgene or by the process of selection in G418. The morphology of pcDNA:IGF-1 transduced clones, for example, c14 and c62, was indistinguishable from that of control cells including IGF-1 treated RPE cells and RPE clones resistant to G418 but not expressing a GFP:IGF-1 fusion protein. The clones expressing the IGF-1 transgene were epithelial in nature, grew in a relatively geometric pattern, and were contact inhibited in vitro.

During continued culture, the appearance of IGF-1 transduced clones remained similar to that of control RPE cells. Some mild variability in morphology was seen in both control and transfected cultures before the cells reached confluence. The enhanced growth characteristics induced by IGF-1 transgene expression (described below) were not associated with any apparent changes in the cell appearance over eight passages in vitro.

### Example 8

### Expression of IGF-1 Transgene in RPE Cells

Quantitative differences in the transcription of the transgene between clone c62 and controls were determined by real-time RT-PCR. The amplification threshold (C_{T}) corresponding to the level of intrinsic IGF-1 transcription in control RPE cells occurred at a mean cycle of 30.23 (standard deviation±0.09). The amplification threshold of IGF-1 in clone c62 occurred at a mean C_{T} value of 22.37 (s.d.±0.06). The difference in C_{T} for clone c62 vs. control RPE was 7.8, demonstrating a greater than 200-fold increase in the level of IGF-1 transcript in this clone, relative to naïve RPE cells (2^{7.8}). The contribution of the transgenic mRNA to the differences seen in the level of IGF-1 transcripts was more clearly demonstrated by semi-quantitative RT-PCR. The C_{T} value for β-actin did not vary between RNA samples (control RPE; 26.15±0.10 vs. c62; 26.45±0.03), confirming that the increase in IGF-1 gene expression was clone-specific.

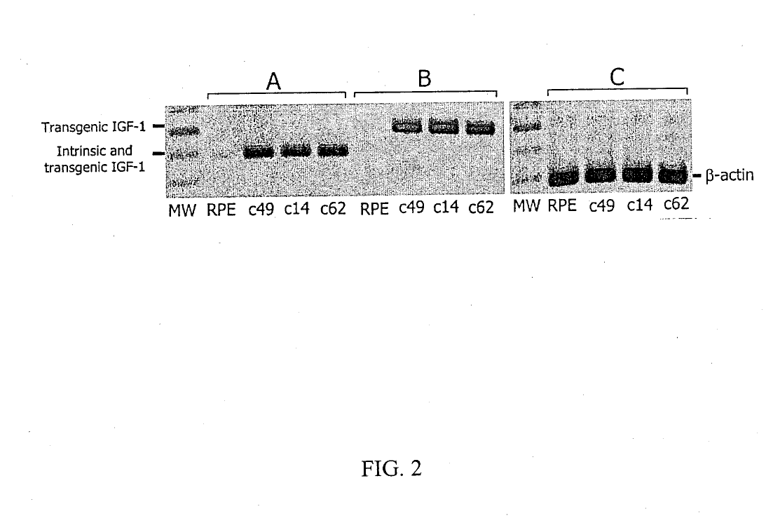

FIG. 2 shows results of semi-quantitative RT-PCR of IGF-1 message in control and transfected RPE cells. As seen in FIG. 2A, a very low level of intrinsic IGF-1 message was present in control RPE cells, whereas a high level of IGF-1 message was detectable in transfected clones c49, c14 and c62. The band seen in FIG. 2A is due to amplification of both the transgenic message and the intrinsic message by primers 1 and 2. Amplification of the transgenic message (primers 2 and 3, FIG. 2B) showed the same level of product as with primers 1 and 2. This result confirmed that the majority of the IGF-1 message present is transgenic. The specificity of primers 2 and 3 for the transgenic IGF-1 mRNA was confirmed by the absence of an amplification product in untransduced RPE cells (FIG. 2B). Equivalent loading of the samples was demonstrated by equal levels of amplified β-actin message in each sample (FIG. 2C).

The results of the RT-PCR and qRT-PCR studies showed low levels of intrinsic IGF-1 message in RPE cells grown under the low serum tissue culture conditions used in these studies. The absolute level of IGF-1 expression in naïve RPE cells was approximately 0.01 picograms per microgram of total RNA. Conversely, the CMV promoter in the plasmid vector directed the transcription of high levels of transgenic IGF-1 MRNA in transduced clones under the same culture conditions. For example, transgenic IGF-1 message was present in clone c62 at >1 picogram per microgram of total RNA. Real-time RT-PCR curves of the constitutively expressed β-actin gene from the same samples were not elevated and demonstrated that the increase in expression was fusion gene-specific.

### Example 9

### Fusion Protein Synthesis and Secretion

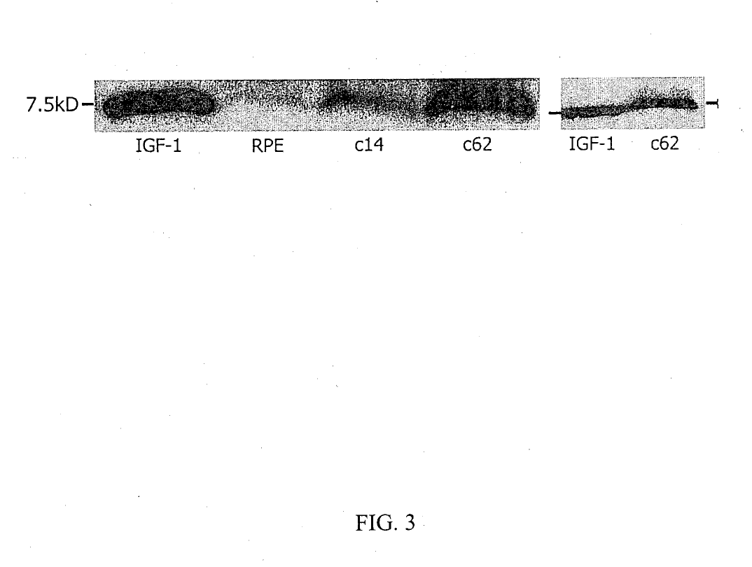

Fusion protein synthesis. Referring to FIG. 3, Western blots of total cellular protein from the clones demonstrated a markedly increased amount of IGF-1 fusion protein in the transfected cells. High levels of transgenic message in transfected clones were correlated with moderate-to-high levels of IGF-1 fusion protein synthesis, relative to intrinsic IGF-1 expression. FIG. 3 shows results of Western blot analysis of IGF-1 protein synthesis by two transfected clones, i.e., c14 and c62, relative to such synthesis by untransfected control RPE cells. Although clones c14 and c62 showed similar levels of IGF-1 transcript, it can be seen that clone c62 synthesized significantly more fusion protein. This finding demonstrates that the efficiency of translation can vary from clone to clone. Also, differences in the control of protein degradation may vary among transfected clones.

Fusion protein secretion. For this analysis, secretion of the IGF-1 fusion protein into the tissue culture media was determined by nickel column chromatography and Western blotting using methods described above. The secreted IGF-1 protein was selectively bound and eluted from a nickel column prior to Western blotting, confirming the presence of the His₆-epitope on the protein.

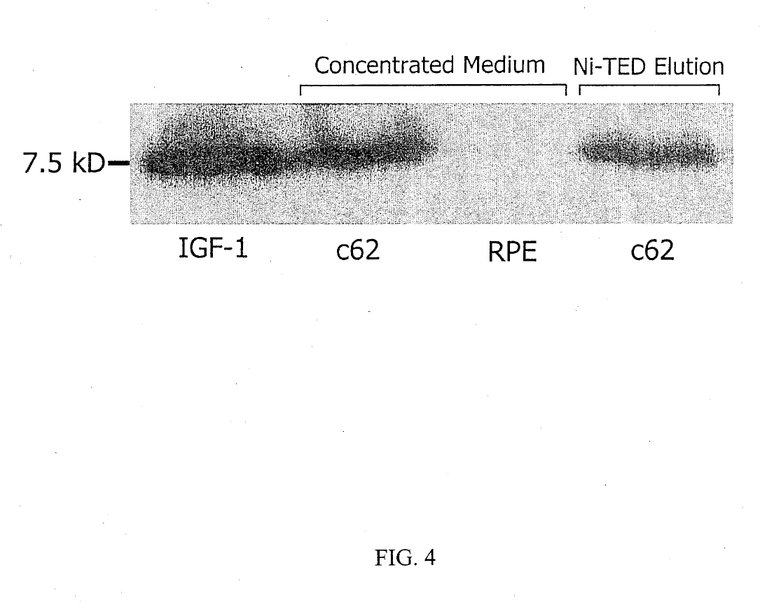

As shown in FIG. 4, fusion protein was detected in concentrated tissue culture media conditioned by clone c62, but was undetectable in concentrated tissue culture media conditioned by naïve RPE cells. The quantitative increase in IGF-1 protein present in the transfected RPE cells was correlated with increased levels of IGF-1 secretion into the media.

### Example 10

### IGF-1 Transfection Promotes RPE Cell Proliferation In Vitro

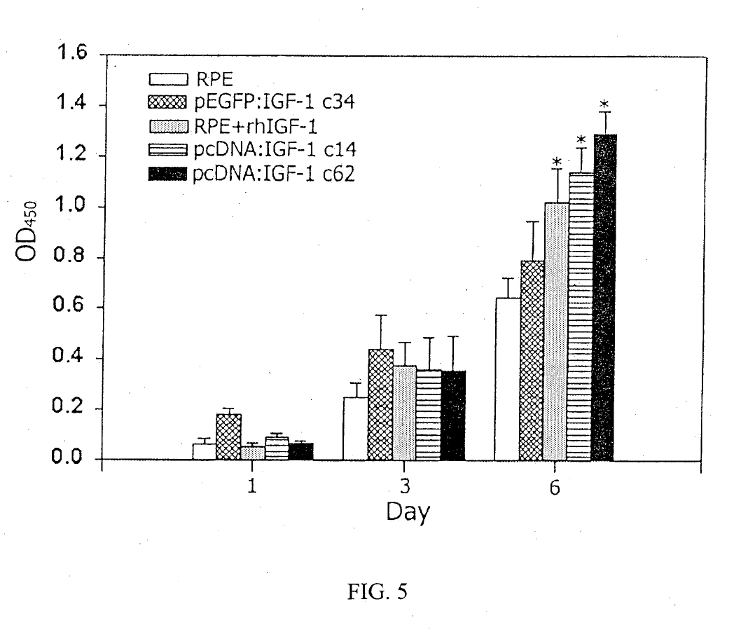

In vitro growth characteristics of clones c14, and c62, were compared with those of positive and negative control cultures using methods described above. As shown in FIG. 5, six days after plating, there was a statistically significant increase in the growth of both clones c62 and c14 (P<0.002, paired samples, t-test), compared with naïve RPE controls. The increased growth rate paralleled an increase in the proliferation of untransduced RPE cells cultured in the presence of recombinant IGF-1 protein (100 ng/ml) (P<0.035), and demonstrated dose-dependent biological activity of the secreted fusion protein. There was no increase in the growth rate of a transduced G418-resistant clone expressing undetectable levels of a GFP fusion protein, compared to naïve RPE controls (c34, FIG. 5).

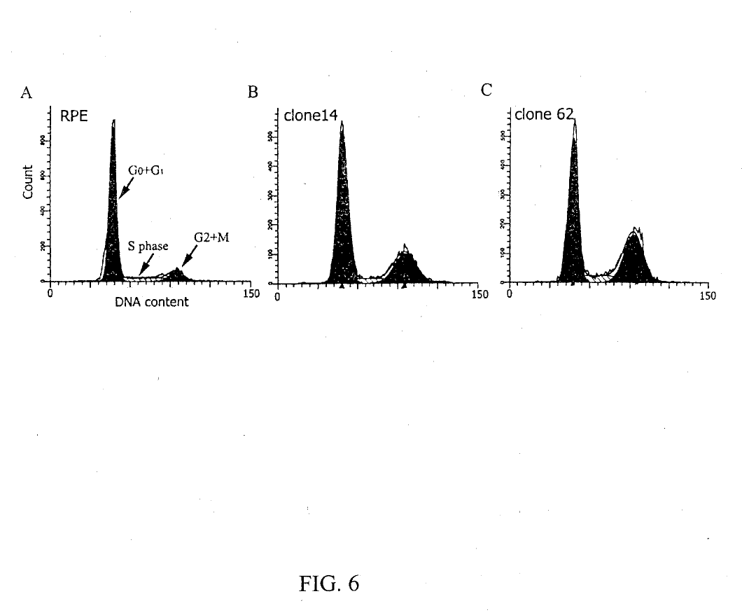

Flow cytometry was used to examine progression of the IGF-1-transduced clones and naïve control RPE cells through the G₀-G₁ phase of the cell cycle after synchronization. The percentage of cells in each phase of the cell cycle (i.e., G₀-G₁, S-phase, and G₂-Mitosis), was determined at 27 hours after release from serum starvation as described above. Referring to FIG. 6, IGF-1 transduced RPE cells demonstrated enhanced cell cycling under low serum conditions after synchronization compared to naïve controls. The percentage of cells that had traversed G₀-G₁ and entered into S-phase or G₂-Mitosis was significantly higher after 27 hours in clones c14 and c62, compared to naïve controls (c14, P<0.044; c62 P<0.027, paired samples, t-test). The RPE cells used in these studies were grown in low serum to minimize the effects of exogenous serum growth factors on RPE cell proliferation. Under these conditions, expression of the IGF-1 transgene clearly resulted in increased recruitment of RPE cells into the proliferative phase of the cell cycle. The observed recruitment was IGF-1 dose-dependent and is consistent with the biological role of IGF-1 in stimulating cellular mitogenesis.

The results showed that proliferation of IGF-1-transduced RPE cells was greater than that of naïve RPE cells receiving direct IGF-1 stimulation by supplementation of the culture media (FIG. 5). This difference in biological response may be due to pulsed versus continuous stimulation by IGF-1. IGF-1 (100 ng/ml) was supplemented in the media of treated cells on days 1 and 3 of the experiment. Conversely, transduced cells secreted the hormone continuously in vitro. This result indicated that continuous delivery of IGF-1 to contiguous cells may be more effective in stimulating cell proliferation than exogenous supplementation of the media with the hormone. Under the latter conditions, degradation and consumption of the hormone would be expected to slowly reduce the level of active hormone in the media over time.

Results of the proliferation assays also demonstrated a direct correlation between the level of IGF-1 fusion protein and the enhanced cell cycle kinetics and cell proliferation rates seen in transduced clones. IGF-1 fusion protein synthesis was seen to enhance cell proliferation in a quantitative and dose-dependent fashion in the clones. These findings provide evidence that the IGF-1 fusion protein is biologically active and mitogenic in RPE cells, and that transgenic synthesis of the protein modifies the phenotype of the transduced clone by imparting an enhanced proliferative capacity under low serum conditions in vitro. This provides strong evidence that the IGF-1 fusion protein plays a direct role in enhancing the proliferative potential of the transduced RPE cells through a paracrine hormonal effect. An autocrine effect from increased levels of intracellular IGF-1 may also be involved.

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A method of increasing synthesis or secretion of a growth factor protein by a RPE cell, the method comprising the steps of:
(a) providing a RPE cell; and
(b) transducing said cell with an expression vector comprising a growth factor-encoding nucleic acid in an amount effective to increase synthesis or secretion of said growth factor protein.

2. The method of claim 1, wherein said growth factor protein is selected from the group consisting of IGF-1, bFGF, aFGF, CNTF, PDGF, and BDNF.

3. The method of claim 1, wherein said growth factor protein is human IGF-1.

4. The method of claim 1, wherein said growth factor protein is a fusion protein.

5. The method of claim 4, wherein said fusion protein comprises an epitope tag.

6. The method of claim 5, wherein said fusion protein comprises a His₆ tag.

7. The method of claim 6, wherein said fusion protein is His₆-tagged human IGF-1.

8. The method of claim 4, wherein said fusion protein comprises a reporter protein.

9. The method of claim 8, wherein said reporter protein is selected from the group consisting of GFP, luciferase and β-galactosidase.

10. The method of claim 8, wherein said fusion protein is IGF-1 tagged with GFP.

11. The method of claim 1, wherein said gene expression vector comprises an inducible promoter.

12. An expression vector comprising a growth factor-encoding nucleic acid, wherein said vector directs the production of an expressed growth factor fusion protein.

13. The vector of claim 12, wherein the fusion protein comprises human IGF-1.

14. The vector of claim 12, wherein the vector is a plasmid.

15. A kit for gene transduction of a cell, said kit comprising at least one expression vector comprising a nucleic acid encoding a human growth factor protein fused with a reporter protein, or a nucleic acid encoding a human growth factor protein fused with an epitope tag, and instructions for use.

16. The kit of claim 15, wherein the at least one expression vector directs expression of a human IGF-1 protein linked to GFP reporter protein.

17. The kit of claim 15, wherein the at least one expression vector directs expression of a human IGF-1 protein linked to a His₆ epitope tag.

18. A retinal cell transduced with the vector of claim 12.

19. The transduced retinal cell of claim 18, wherein the cell is a RPE cell.

20. The cell of claim 19, wherein said cell is a human cell.

21. A method of treating dysfunction or injury of a RPE cell, said method comprising transducing said cell with an expression vector that expresses a growth factor protein in an amount sufficient to improve or cure said dysfunction or injury.

22. The method of claim 21, wherein the dysfunction affecting the RPE cell is caused by cancer, viral infection, diabetes, hereditary RPE dystrophy, AMD, retinitis pigmentosa, or drug-induced toxicity.

23. The method of claim 21, wherein the dysfunction affecting the RPE comprises inability to divide, or commitment to programmed cell death.

24. The method of claim 21, wherein said injury of the RPE cells is caused by abrasions, ocular trauma, surgical and laser procedures, or retinal detachment.

25. A method of treating a retinal disease or condition, said method comprising the steps of:
(a) providing a subject having or at risk for developing a retinal disease or condition;
(b) providing an expression vector comprising a growth factor-encoding nucleic acid; and
(c) administering to said subject an amount of said vector sufficient to ameliorate or cure said disease.

26. The method of claim 25, wherein the retinal disease or condition is selected from the group consisting of cancer, viral infection, diabetic retinopathy, hereditary RPE dystrophy, AMD, retinitis pigmentosa, drug-induced toxicity, macular commotio, surgical injury, and laser-induced injury.

27. The method of claim 20, wherein said growth factor is human IGF-1.
